(19) United States
(12) Plant Patent Application Publication
(10) Pub. No.: US 2003/0046741 P1
(43) Pub. Date: Mar. 6, 2003

(54) Chrysanthemum named ' White Elite Reagan'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/902,767

(22) Filed: Jul. 12, 2001

(51) Int. Cl. A01H 5/00

(52) U.S. Cl. PLT/294

(57) ABSTRACT

A Chrysanthemum plant named ‘White Elite Reagan’ characterized by its medium sized blooms with white ray-florets and yellow-green disc florets.

### RELATED CULTIVARS

‘White Elite Reagan’ is related to ‘White Reagan’ (U.S.Plant Pat. No. 8744)) and to the co-pending applications of ‘Cream Elite Reagan’ and ‘Sunny Elite Reagan’. The cultivar ‘White Elite Reagan’ is a natural mutant of ‘White Reagan’. The co-pending ‘Cream Elite Reagan’ and ‘Sunny Elite Reagan’ are natural mutants from ‘White Elite Reagan’.

### BACKGROUND OF THE INVENTION

‘White Elite Reagan’ is a product of a breeding and selection program that had the objective of finding mutants of ‘White Reagan’. The new plant of the present invention comprises a new and distinct cultivar of chrysanthemum plant that is a natural occuring sport of a parent chrysanthemum named ‘White Reagan’ (U.S. Plant Pat. No. 8744). A comparison with Parent chrysanthemum ‘White Reagan’ is also made in this application. The new cultivar was discovered as a whole plant mutation in September 1995 by Rob Noodelijk in a controlled environment (greenhouse) in Rijsenhout Holland. The first act of asexual reproduction of ‘White Elite Reagan’ was accomplished when vegetative cuttings were taken from the initial selection in November 1995 in Rijsenhout Holland.

### SUMMARY OF THE INVENTION

The present invention is a new and distinct variety of chrysanthemum bearing medium sized blooms with white ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

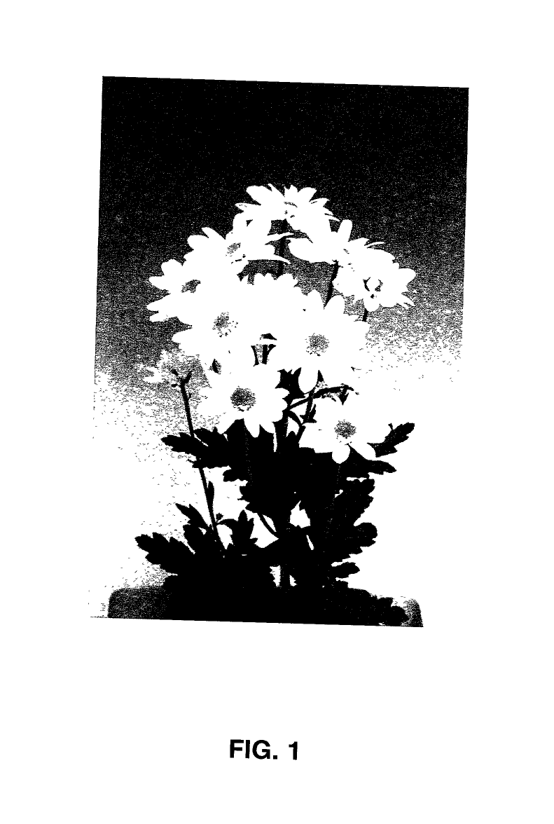

FIG. 2 shows the various stages of bloom of the new cultivar.

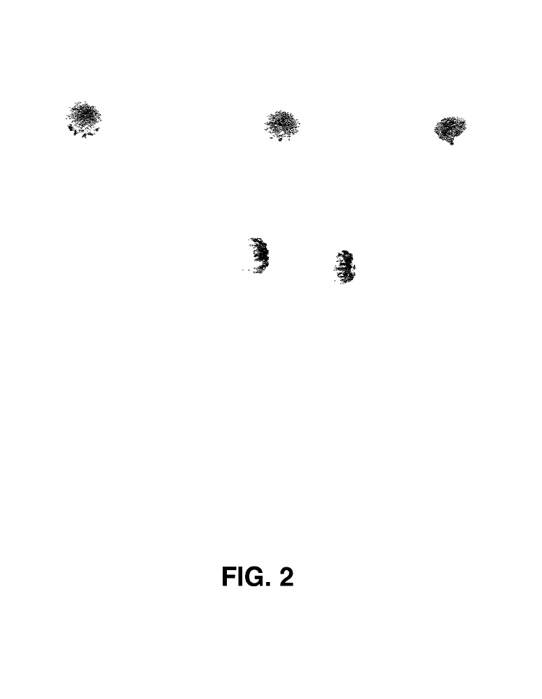

FIG. 3 shows the foliage of the new cultivar.

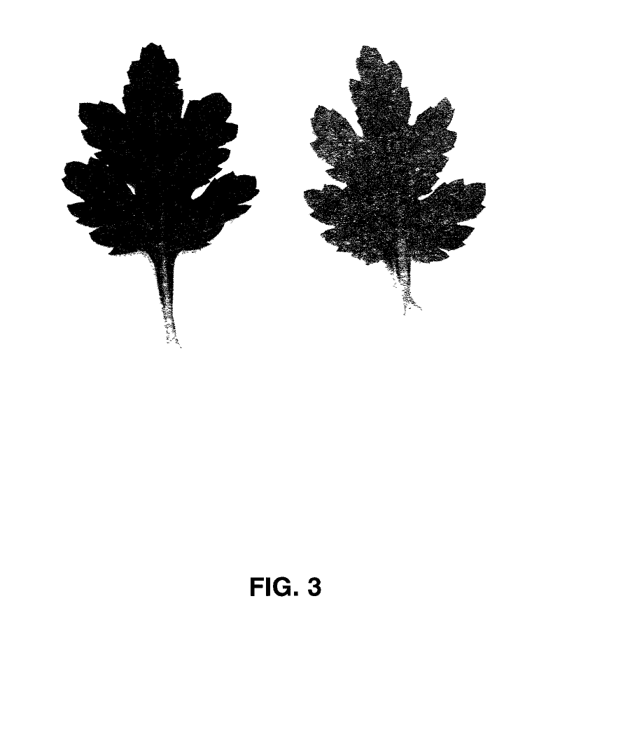

### DESCRIPTION OF THE INVENTION

This new variety of chrysanthemum is of the botanical classification dendranthema grandiflora. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 49 days. After the long day period to flowering growth retardants were applied 2 to 3 times in an average dose of 2.5 gram/liter water. No tests were done on disease or insect resistance or susceptibility. This new variety produces medium sized blooms with white ray-florets and yellow-green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘White Elite Reagan’ can be planted without assimilation lightning (high pressure sodium lamps) between week 50 and week 40 of the next year under greenhouse conditions in Holland. With assimilation light (minimum level 2500 lux) it can be planted year round under greenhouse conditions in Holland.

From the cultivars known to inventor the most similar existing cultivar in comparison to ‘White Elite Reagan’ is ‘White Reagan’ and ‘White Reagan Mundo’. When ‘White Reagan’ and ‘White Elite Reagan’ are being compared the following differences and similarities are noticed: The differences of ‘hite Reagan’ and ‘White Elite Reagan’ are: (1) Flower color. The flower of ‘White Elite Reagan’ is more pure white. (2) Color of the buds. The color of the buds of ‘White Elite Reagan’ is also more pure white. (3) Color of the disc flowers. The disc of ‘White Elite Reagan’ is greener. (4) The flower disc of ‘White Elite Reagan’ is smaller. (5) Flower size. The flower of ‘White Elite Reagan’ is a little smaller. (6) Flower shape. The ray-florets of ‘White Elite Reagan’ are more pointed and are more upright (7) the growth rate. The growth of ‘White Elite Reagan’ is more vigorous. (8) Response time. The response time of ‘White Elite Reagan’ is several days shorter. For these characteristics ‘White Elite Reagan’ has been selected out of ‘White Reagan’. All other characteristics of ‘White Reagan’ and ‘White Elite Reagan’ are very similar.

‘White Reagan Mundo’ has the same flower shape as ‘White Elite Reagan’. The flower disc however is not so small. The growth is less vigorous instead of more vigorous. The spray formation is more spreading, with a bigger angle of the peduncles with the stem instead of more upright for ‘White Elite Reagan’ and the most essential difference: ‘White Elite Reagan’ does not have a low temperature tolerance as ‘White Reagan Mundo’.

The following is a description of the plant and characteristics that distinguish ‘White Elite Reagan’ as a new and distinct variety.

The color designations are taken from the plant itself. Accordingly, any discrepancies between the color designations and the colors depicted in the photographs are due to photographic tolerances. The color chart used in this description is: The Royal Horticultural Society color chart, edition 1995.

### TABLE 1

### Botanical Description of CULTIVAR ‘White Elite Reagan’

Bud

Size.—Medium, cross-section +/−1.0 cm height +/−0.8 cm

Outside color.—White 155 A

Involucral bracts.—2 rows, length 7 mm, width 3 mm

Involucral bracts among disc-florets.—Not present

Involucral bracts color.—Green 138 B

Bloom

Type.—Single

Height.—Flat

Size.—Medium

Fully expanded.—6.0-7.0 cm

Number of blooms per single stem.—Average of 11 to 13

Seeds.—Not produced

Performance (blooming period).—26-28 days

Fragrance.—Typical chrysanthemum

Color

Center of the flower (disc-florets).—Immature yellow-green 144 A, mature yellow-green 144 A in the center to yellow 3 A along the border

Color of upper surface of the majority of the ray-florets.—White 155 D

Color of the lower surface of the majority of the ray-florets.—White 155 D

Tonality from distance.—A spray mum with white flowers and a yellow-green disc

Discoloration to color.—No discoloration

Ray florets

Texture.—Upper and under side smooth

Number.—22-24

Cross-section.—Flat, sometimes convex

Longitudinal axis of majority.—Straight, sometimes reflexing

Length of corolla tube.—Very short to short

Ray-floret length.—3.5 cm

Ray-floret width 1.2 cm

Ratio length/width.—Low to medium

Shape of tip.—Pointed

Disc florets

Disc diameter.—Medium to small, 1.0-1.2 cm

Distribution of disc florets.—Numerous, clearly visible at all stages of flowering

Type.—Tubular

Color.—Yellow-green 144 A

Receptacle shape.—Conical raised

Reproductive Organs

Stamen (present in disc florets only).—Yellow-green, thin 3 mm in length

Stamen color.—Yellow-green 150 B

Pollen.—Appears at a very late stage of flowering,

Pollen color.—Close to yellow 12 A

Styles (present in both ray and disc florets).—Green, thin

Style color.—Green 143 B

Style length.—4 mm

Stigmas.—Yellow-green

Stigma width.—1 mm

Ovaries.—Enclosed in calyx.

Plant

Form.—A spray mum meant for erect culture.

Growth habit.—Upright.

Growth rate.—Vigorous.

Height.—105-130 cm

Internodes 3-3.5 cm

Spray formation.—Corymbiform (funnel-shaped, with a little round to flat umbel on top)

Peduncle length.—Near the top 14 cm, near the middle 21 cm, near the bottom 26 cm.

Peduncle color.—Green 138 B.

Peduncle, attachment.—Good, not brittle.

Peduncle, angle with stem.—Small, 30°.

Stem color.—Green 144 A.

Stem strength.—Medium.

Stem brittleness.—Absent.

Stem anthocyanin coloration.—Absent.

Flowering response(photo-periodic controlled crop, not natural season).—49 days.

Foliage

Color.—Upper side green 137 A Under side green 147 B.

Size.—Medium; length +/−12 cm, width +/−8 cm.

Quantity (number per stem).—22-28.

Shape.—Ovate.

Texture upper side.—Scabrous.

Texture under side.—Slightly pubescent.

Venation arrangement.—Pinnate.

Shape of the margin.—Lobed.

Shape of base of sinus between lateral lobes.—Round.

Margin of sinus between lateral lobes.—Converging.

Shape of base.—Truncate.

Apex.—Mucronate

TABLE 2
Differences with the comparison Varieties

| | ‘White Elite | ‘White | ‘White Reagan |
|---|---|---|---|
| | Reagan’ | Reagan’ | Mundo’ |
| Color of the buds | White 155 A | Yellow 4 D | Yellow 4 D |
| Flower disc size | 1.0-1.2 cm | 1.5 cm | 1.3-1.5 cm |
| Growth rate | Vigorous | Medium | Medium |
| Plant height | 105-130 cm | 100-125 cm | 100-115 |
| Peduncle angle with | 30° | 30-35° | 40° |
| the stem | | | |
| Required minimum | 17° C. | 17° C. | 14° C. |
| night temperature | | | |

I claim:

1. A new and distinct variety of chrysanthemum plant as described and illustrated.
